(19) United States
(12) Patent Application Publication
(10) Pub. No.: US 2018/0078339 A1
(43) Pub. Date: Mar. 22, 2018

(54) METHODS AND DEVICES FOR STIMULATING CELL PROLIFERATION

(71) Applicant: Advanced Orthodontics And Education Association, LLC, Ossining, NY (US)

(72) Inventors: Richard Johnson, Briarcliff Manor, NY (US); Bryce A. Way, San Jose, CA (US)

(73) Assignee: Advanced Orthodontics And Education Association, LLC, Ossining, NY (US)

(21) Appl. No.: 15/672,981

(22) Filed: Aug. 9, 2017

Related U.S. Application Data
(63) Continuation-in-part of application No. PCT/US2017/019767, filed on Feb. 27, 2017.
(60) Provisional application No. 62/300,705, filed on Feb. 26, 2016.

(51) Int. Cl. A61C 7/00; A61C 7/08; A61K 35/32; A61K 35/33

(52) U.S. Cl. CPC A61C 7/008; A61C 7/08; A61C 7/00; A61K 35/33; A61K 35/32

(57) ABSTRACT

Embodiments of the present disclosure are directed to devices and methods for stimulating cell proliferation. In one implementation, a method for increasing cell proliferation is provided. The method includes providing a vibrational dental device configured to vibrate at a frequency higher than about 80 Hz. The method also includes mechanically stimulating, using the vibrational dental device, cells for less than about 20 minutes daily over a period of time. The number of the cells at the end of the period of time is increased. The cells may include one of human osteoblasts and fibroblasts.

### BACKGROUND

### Technical Field

The present disclosure generally relates to dental devices and methods of use. More particularly, and without limitation, the disclosed embodiments relate to systems, devices, and methods for stimulating cell proliferation in the region of the alveolar process of the maxilla and mandible, including the periodontal ligament, using vibration.

### Background Description

Mechanical vibration may enhance musculoskeletal properties. For example, some studies suggested that low-intensity mechanical vibrations may stimulate bone formation or mitigate the degradation of the intervertebral disc in rats. However, the biomolecular mechanisms for such enhancement effects have not yet been elucidated. Some studies suggested that mechanical vibration may enhance differentiation of human bone marrow mesenchymal stem cells or periodontal ligament stem cells. But discrepancies and unpredictability exist in literature as to the effects of mechanical vibration on cell proliferation. For example, previous studies have demonstrated no effects or either increased or decreased proliferation after cyclic vibration treatment. See Zhang, C., et al. (2012). Effects of mechanical vibration on proliferation and osteogenic differentiation of human periodontal ligament stem cells. Archives of Oral Biology, 57(10), 1395-1407.

The unpredictability in the art has manifested itself in the experimental data in the prior art. Some studies have suggested vibrations at frequencies ranging from 15 Hz to 90 Hz may be more anabolic to bone formation. See Zhang, C., et al. (2012). Most of the previous studies subjected cells or animals to vibration treatments for a certain number of vibration bouts per day, each bout lasting for a duration ranging from 15 minutes to 60 minutes. See Judex, S., et al. (2015). Modulation of bone's sensitivity to low-intensity vibrations by acceleration magnitude, vibration duration, and number of bouts. Osteoporosis International, 26(4), 1417-1428; see also Pongkitwitoon, S., et al. (2016). A recent study further suggested that increasing the bout duration to an even longer period (30 or 60 minutes) would positively influence bone formation rates in mice. Cytoskeletal Configuration Modulates Mechanically Induced Changes in Mesenchymal Stem Cell Osteogenesis, Morphology, and Stiffness. Scientific Reports, 6(1). doi:10.1038/srep34791.

It is recently hypothesized that mechanical vibration may promote periodontal regeneration and periodontal tissue remodeling during and following orthodontic tooth movement. However, variables of mechanical vibration to be used for modulating bone biology so as to effectively accelerate orthodontic tooth movement remain to be determined.

### SUMMARY

The embodiments of the present disclosure include systems, devices, and methods for stimulating cell proliferation in periodontal tissues, including the alveolar processes and periodontal ligament. Advantageously, the exemplary embodiments allow cells belonging to the connective-tissue cell family, such as human osteoblasts in alveolar bone and periodontal ligament fibroblasts, to proliferate after relatively short vibration treatments, thereby improving the efficiency and effectiveness of orthodontic treatments.

According to an exemplary embodiment of the present disclosure, a method for increasing cell proliferation is described. The method includes providing a vibrational dental device that is capable of vibrating at a frequency higher than about 80 Hz. The method also includes mechanically stimulating, using the vibrational dental device, cells for a treatment period of less than about 20 minutes daily over a period of time. The treatment period can be, for example, less than about 20 minutes, 15 minutes, 10 minutes, 6 minutes, 5 minutes, 4 minutes, or less. It is contemplated that in other embodiments the treatment period could be any value within the range of about 1 minute and 19 minutes daily, and that the daily total treatment period could be formed of a plurality of treatment sessions contributing to the daily total treatment period. In one exemplary embodiment, the daily total treatment period is about 5 minutes. The cells may include human osteoblasts and/or human fibroblasts. The method further includes increasing the number of the cells at the end of the period of time. The vibration frequency can be, for example, from about 110 Hz to about 120 Hz, from about 100 Hz to about 110 Hz, from about 90 Hz to about 100 Hz, or from about from about 80 Hz to about 90 Hz. It is contemplated that in other embodiments the frequency could be any value within the range of about 80 Hz and about 120 Hz, and that the vibration frequency could be adjusted during a treatment period. In one exemplary embodiment, the vibration frequency is about 100 Hz.

According to a further exemplary embodiment of the present disclosure, a method for accelerating orthodontic tooth movement is described. The method includes providing a mouthpiece of a vibrational dental device to be clamped by the user's teeth, for example, between the occlusal surfaces of a user's teeth. The method further includes mechanically stimulating, using the vibrational dental device, cells of the user for a treatment period of less than about 20 minutes daily at a frequency higher than 80 Hz over a period of time. The treatment period can be, for example, less than about 20 minutes, 15 minutes, 10 minutes, 6 minutes, 5 minutes, 4 minutes, or less. It is contemplated that in other embodiments the treatment period could be any value within the range of about 1 minute and 19 minutes daily, and that the daily total treatment period could be formed of a plurality of treatment sessions contributing to the daily total treatment period. In one exemplary embodiment, the daily total treatment period is about 5 minutes. The cells may include at least one of osteoblasts in alveolar bone and periodontal ligament fibroblasts. The vibration frequency can be, for example, from about 110 Hz to about 120 Hz, from about 100 Hz to about 110 Hz, from about 90 Hz to about 100 Hz, or from about from about 80 Hz to about 90 Hz. It is contemplated that in other embodiments the frequency could be any value within the range of about 80 Hz and about 120 Hz, and that the vibration frequency could be adjusted during a treatment period. In one exemplary embodiment, the vibration frequency is about 100 Hz.

According to a yet further exemplary embodiment of the present disclosure, a dental device for increasing cell proliferation is described. The dental device includes means for mechanically stimulating cells at a frequency higher than 80 Hz for a treatment period of less than about 20 minutes daily over a period of time, and further increasing the number of the cells at the end of the period of time. The cells may include human osteoblasts and/or human fibroblasts. The treatment period can be, for example, less than about 20 minutes, 15 minutes, 10 minutes, 6 minutes, 5 minutes, 4 minutes, or less. It is contemplated that in other embodiments the treatment period could be any value within the range of about 1 minute and 19 minutes daily, and that the daily total treatment period could be formed of a plurality of treatment sessions contributing to the daily total treatment period. In one exemplary embodiment, the daily total treatment period is about 5 minutes. The vibration frequency can be, for example, from about 110 Hz to about 120 Hz, from about 100 Hz to about 110 Hz, from about 90 Hz to about 100 Hz, or from about from about 80 Hz to about 90 Hz. It is contemplated that in other embodiments the frequency could be any value within the range of about 80 Hz and about 120 Hz, and that the vibration frequency could be adjusted during a treatment period. In one exemplary embodiment, the vibration frequency is about 100 Hz.

Additional features and advantages of the disclosed embodiments will be set forth in part in the description that follows, and in part will be obvious from the description, or may be learned by practice of the disclosed embodiments. The features and advantages of the disclosed embodiments will be realized and attained by the elements and combinations particularly pointed out in the appended claims. It is to be understood that both the foregoing general description and the following detailed description are examples and explanatory only and are not restrictive of the disclosed embodiments as claimed.

The accompanying drawings constitute a part of this specification. The drawings illustrate several embodiments of the present disclosure and, together with the description, serve to explain the principles of the disclosed embodiments as set forth in the accompanying claims.

### BRIEF DESCRIPTION OF THE DRAWINGS

FIG. 1A is a perspective view of an exemplary vibrational dental device, according to embodiments of the present disclosure.

FIG. 1B is a partial perspective view of the exemplary vibrational dental device of FIG. 1A, according to embodiments of the present disclosure.

FIG. 1C is a partial component view of the exemplary vibrational dental device of FIG. 1, according to embodiments of the present disclosure.

FIG. 2 illustrates the operation of the exemplary oral vibrational device of FIG. 1.

FIGS. 3A-3P each show the measurement of vibration of an exemplary typodont subject to vibration treatment by the exemplary vibrational dental device of FIG. 1 under different testing conditions.

FIGS. 4A-4P each show the measurement of vibration of an exemplary typodont subject to vibration treatment by a commercially available dental device under different testing conditions.

FIG. 5A graphically compares g-force measurements of a typodont with an aligner subject to vibration treatment by the exemplary vibrational dental device of FIG. 1 and an exemplary commercially available dental device.

FIG. 5B graphically compares g-force measurements of a typodont without an aligner subject to vibration treatment by the exemplary vibrational dental device of FIG. 1 and an exemplary commercially available dental device.

FIG. 6 graphically compares mean numbers of osteoblasts over three days of a control group, a first group subject to mechanical vibration by a commercially available dental device, and a second group subject to mechanical vibration by the exemplary vibrational dental device of FIG. 1.

FIG. 7 graphically compares mean numbers of periodontal ligament fibroblasts over three days of a control group, a first group subject to mechanical vibration by the exemplary vibrational dental device of FIG. 1, and a second group subject to mechanical vibration by a commercially available dental device.

FIG. 8 is a flowchart of an exemplary method for increasing cell proliferation, according to embodiments of the present disclosure.

FIG. 9 is a flowchart of an exemplary method for accelerating orthodontic tooth movement, according to embodiments of the present disclosure.

### DETAILED DESCRIPTION

The disclosed embodiments relate to devices, systems, and methods for stimulating cell proliferation. Advantageously, embodiments of the present disclosure can be implemented in an orthodontic treatment for accelerating orthodontic tooth movement.

Osteoblasts and fibroblasts are intimately involved in facilitating tooth movement and thus are typically targeted by techniques that aim at accelerating tooth movement in orthodontic treatments. Different approaches, both pre-clinically and clinically, have been attempted to achieve quicker results, but there are still many uncertainties and undetermined variables towards most of these techniques. For example, some commercially available dental devices, such as AcceleDent Aura™, developed by OrthoAccel® Technologies, Inc., are offered to accelerate orthodontic treatment. Patients are advised to wear such dental devices that mechanically stimulate teeth and braces in the form of micro-vibrations at a frequency of 30 Hz for 20 min per day.

To get the maximum desired results of accelerated orthodontic treatment, further studies are still needed to identify the variables of mechanical vibrational stimulation, such as frequency, strength, and duration. It was discovered that treating cells, such as human osteoblasts and fibroblasts, with mechanical vibration for less than about 20 minutes, for example for about 5 minutes, daily at a frequency higher than about 80 Hz increases the number of cells over a period of time. That a marked improvement of cell proliferation over prior-art treatment methods using a shorter treatment interval is surprising and not suggested by the prior art. Various aspects of the present disclosure are developed based on such discovery.

According to an aspect of the present disclosure, a vibrational dental device that vibrates at a frequency higher than about 80 Hz is provided. The vibrational dental device includes a mouthpiece and a motor connected to the mouthpiece. The mouthpiece is configured to be provided between the occlusal surfaces of a user's teeth so as to be clamped by the user's teeth. The motor is configured to vibrate the mouthpiece at a frequency higher than about 80 Hz, such as at a frequency between about 100 Hz to about 120 Hz, and with an acceleration magnitude ranging between about 0.03 G and about 0.4 G. When the motor is in operation and when the mouthpiece is clamped between the occlusal surfaces of a user's teeth, the vibrational dental device applies an axial vibratory force on the occlusal surfaces. Clamping the teeth onto the mouthpiece will apply a load to the vibrator, which may have the effect of lowering the vibrational frequency of the mouthpiece measured in free air, as described below.

In some embodiments, the vibrational dental device may further include a sensor configured to detect the vibration variables of the device, such as frequency and acceleration magnitude. When the mouthpiece of the vibrational dental device is clamped between the occlusal surfaces of a user's teeth, the sensor may detect the vibration variables proximate to the occlusal surfaces of the user's teeth. In some embodiments, the sensor is a piezoelectric sensor.

According to another aspect of the present disclosure, a method for increasing cell proliferation is provided. The method includes mechanically stimulating, using an exemplary vibrational dental device, cells for less than about 20 minutes, for example for about 5 minutes, daily at a frequency higher than about 80 Hz over a period of time. The cells may include at least one of human osteoblasts and fibroblasts. The period of time may extend for a couple of days up to a number of months, at the end of which, the number of the cells increases. As described herein, an increase of cell proliferation or an increase of the number of cells can be represented by an increase of cell density.

According to another aspect of the present disclosure, a method for accelerating orthodontic tooth movement is described. The method includes providing the mouthpiece of the vibrational dental device between the occlusal surfaces of a user's teeth to be clamped by the user's teeth. The method further includes mechanically stimulating, using the vibrational dental device, cells of the user, including at least one of osteoblasts in alveolar bone and periodontal ligament fibroblasts, for less than about 20 minutes, for example for about 5 minutes, at a frequency higher than 80 Hz daily over a period of time.

In some embodiments, the method further includes applying an axial vibratory force on the occlusal surfaces by the vibrational dental device. In some embodiments, the vibrational frequency and/or the acceleration magnitude generated by the vibrational dental device may be adjusted. Such adjustment may depend on one or more factors, such as the speed of tooth movement and/or user's reported comfort level.

Reference will now be made in detail to embodiments and aspects of the present disclosure, examples of which are illustrated in the accompanying drawings. Where possible, the same reference numbers will be used throughout the drawings to refer to the same or like parts.

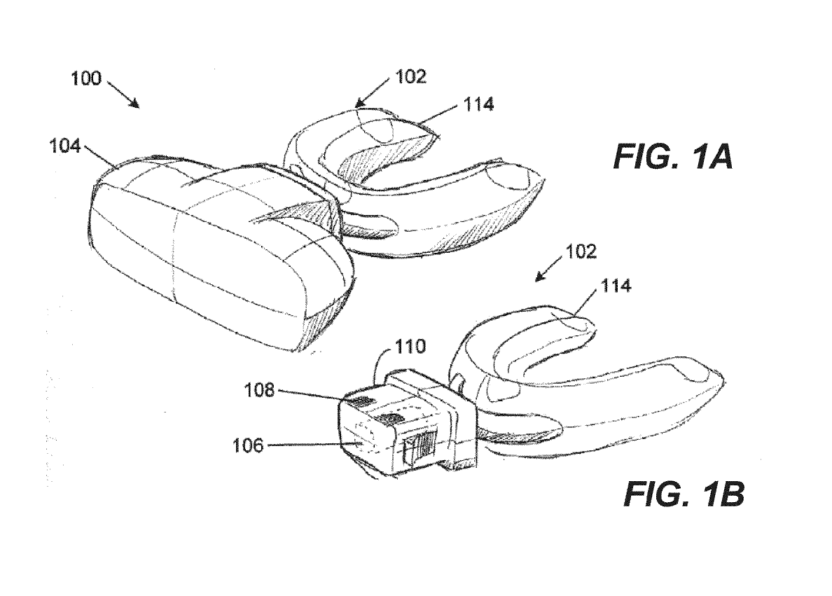

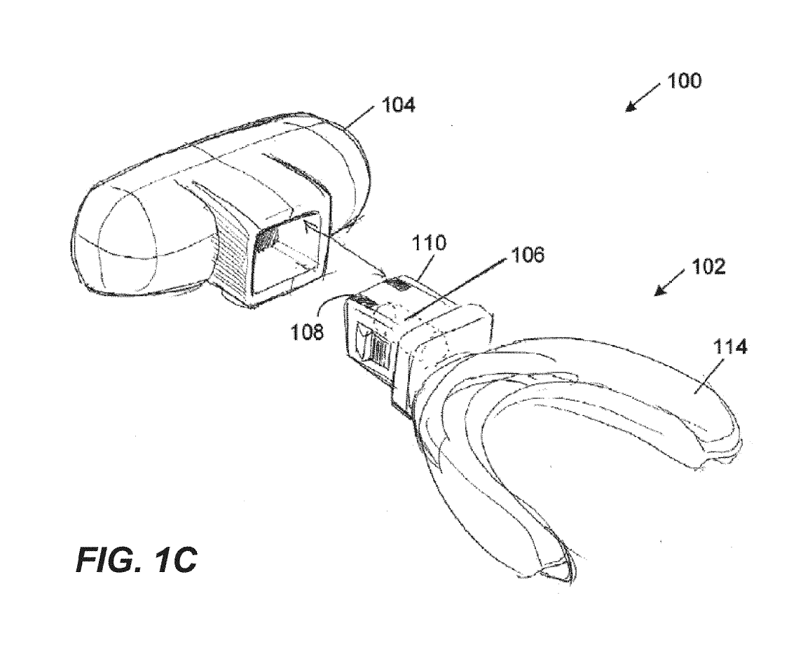

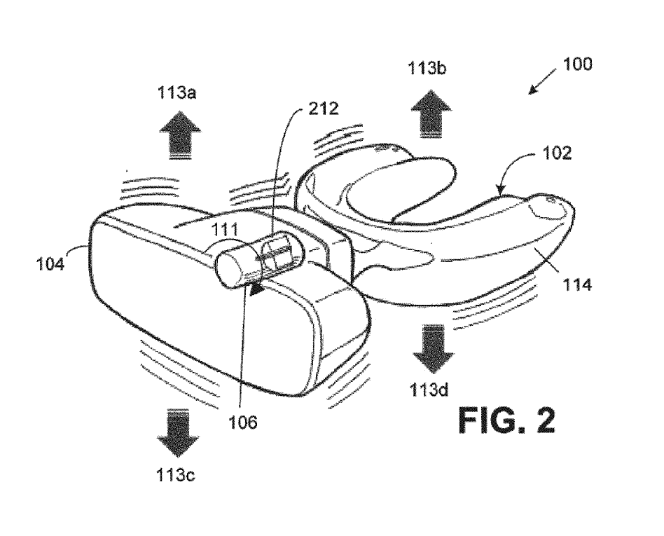

FIG. 1A is a perspective view of an exemplary vibrational dental device 100. FIG. 1B is a partial perspective view of vibrational dental device 100. FIG. 1C is a partial component view of vibrational dental device 100. As shown in FIGS. 1A-1C, vibrational dental device 100 includes a mouthpiece 102, a base 104, and a motor 106. Mouthpiece 102 is removably attached to base 104. Mouthpiece 102 includes a biteplate 114 and a mouthpiece extension 110 configured to connect with base 104. In some embodiments, mouthpiece 102 and/or biteplate 114 can be configured to engage some or all of a user's teeth. For example, in the exemplary embodiments shown in FIGS. 1A-2, mouthpiece 102 and/or biteplate 114 are shaped to engage some or all of a user's teeth. As described herein, the shape of mouthpiece 102 and/or biteplate 114 shown in FIGS. 1A-2 is only exemplary. Mouthpiece 102 and/or biteplate 114 may have a C-shape as depicted, or a customized shape suitable for safe application of vibrational treatment to all or some of a user's teeth. The mouthpiece can be made to apply vibration directly to a user's teeth, or to aligners or other appliances applied to the teeth. Extension 110 may further include contacts 108 that electrically connect base 104 with motor 106.

As shown in FIGS. 1B and 1C, motor 106 is installed in extension 110 of mouthpiece 102. When mouthpiece 102 is attached to base 104, motor 106 resides in base 104. Base 104 further includes electronic circuitries (not shown), including a control circuitry and a power circuitry, for operating motor 106. Motor 106 may be any type of motor that can cause mouthpiece 102 or biteplate 114 to vibrate. For example, motor 106 could be a vibration motor, piezoelectric motor, a linear motor, or an electromagnetic motor. The frequency and/or strength of vibration caused by motor 106 can be adjusted by changing the voltage or current supplied to motor 106 by the electronic circuitries in base 104. For example, the voltage used for operating motor 106 may range from about 0.5 volt to about 4 volts. The current supplied to an exemplary motor 106 may range from about 65 mA to about 100 mA.

Motor 106 may have any suitable mechanical configurations to cause mouthpiece 102 or biteplate 114 to vibrate axially. FIG. 2 illustrates exemplary operation of vibrational dental device 100. As shown in FIG. 2, in one embodiment, motor 106 is a counter-weighted motor with a longitudinal axis parallel to the longitudinal axis of extension 110. Motor 106 may include a counterweight 212 that is off-axis from the longitudinal axis of motor 106. When the motor 106 rotates, as shown by the arrow 111 in FIG. 2, counterweight 212 moves up and down, causing the mouthpiece 102 to vibrate up and down, as shown by the arrows 113a-113d in FIG. 2. Accordingly, when the exemplary mouthpiece 102 is placed in a typodont or between a user's teeth, and when exemplary vibrational dental device 100 is turned on, the vibration of mouthpiece 102 will apply an axial vibratory force on the occlusal surfaces of the teeth. For example, biteplate 114 of mouthpiece 102 may cyclically move axially between the occlusal surfaces of the teeth.

In some embodiments, vibrational dental device 100 may further include one or more sensors (not shown), such as piezoelectric sensors, configured to detect the acceleration magnitude and/or frequency of the vibration of mouthpiece 102. For example, sensors may be placed on the outside or the inside edge of biteplate 114, proximate to the occlusal surfaces of the teeth when mouthpiece 102 is clamped between the occlusal surfaces. The sensors can be electrically connected to the electronic circuitries in base 104. Measurements of the sensors can be fed back to the control circuitry of motor 106 to adjust the acceleration magnitude and/or frequency of motor 106. For example, the detected acceleration magnitude and/or frequency may be compared to a desired acceleration magnitude and/or frequency, and the voltage and/or current supplied to motor 106 can be adjusted based on the comparison.

In some embodiments, motor 106 is configured to vibrate mouthpiece 102 at a frequency higher than about 80 Hz, such as at a frequency between about 100 Hz to about 120 Hz. Motor 106 may be further configured to vibrate mouthpiece 102 at an acceleration magnitude ranging between about 0.03 G and about 0.2 G. As described herein, the vibrational frequency of mouthpiece 120 may vary from the rated “free-air” vibrational frequency of motor 106 due to the amount of biting force or load applied to mouthpiece 102, such as the force used to clamp vibrational dental device 100 in place. For example, when motor 106 is configured to vibrate at a frequency of about 120 Hz, adding biting force or load to mouthpiece 102 may result in a lower vibrational frequency of mouthpiece 102 ranging from about 100 Hz to about 120 Hz.

Examples 1-11 described below illustrate the use of vibrational dental device 100 operating under these variables and its clinically relevant effects.

### Example 1

A simulation was conducted to test and compare the vibration characteristics of a typodont caused by an exemplary embodiment of vibrational dental device 100 and a commercially available dental device, the AcceleDent Aura™. In the simulation setup, the typodont was secured to a metal table. The upper jaw of the typodont was hinged to the lower jaw and capable of opening and closing. Each device was placed in the typodont (between the occlusal surfaces) and held in position by securely mounting a weight of about 0 to about 4 pounds on the upper jaw. The weight simulates the biting force typically applied by a user to clamp the devices in place.

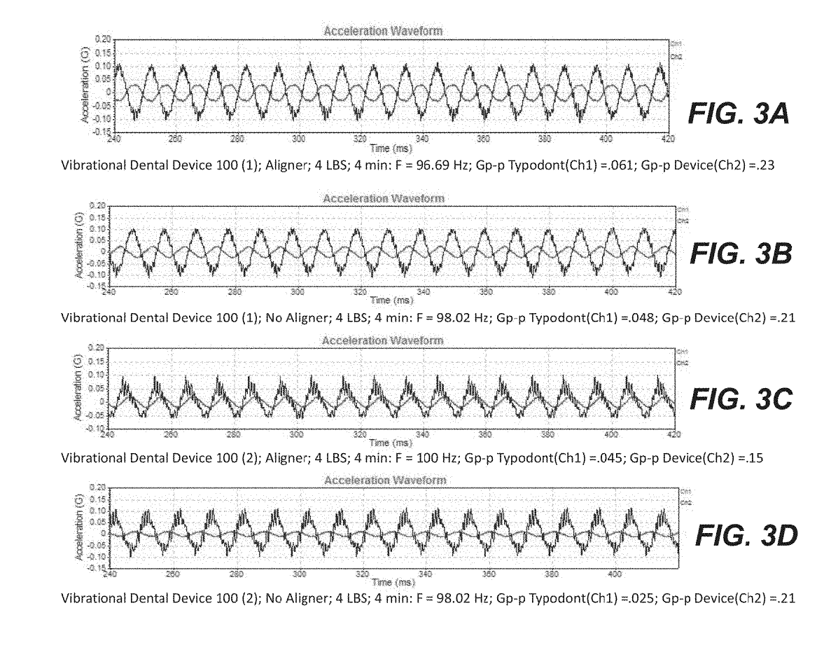

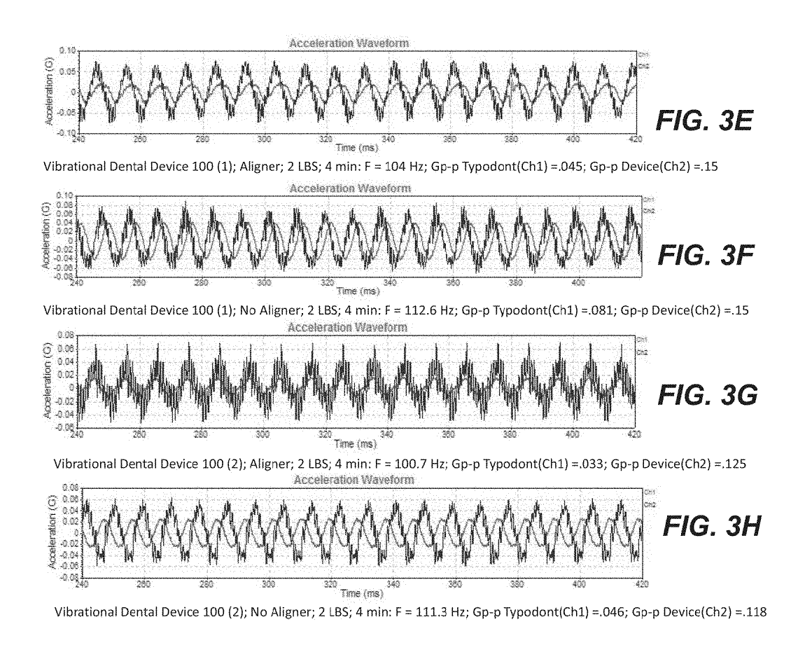

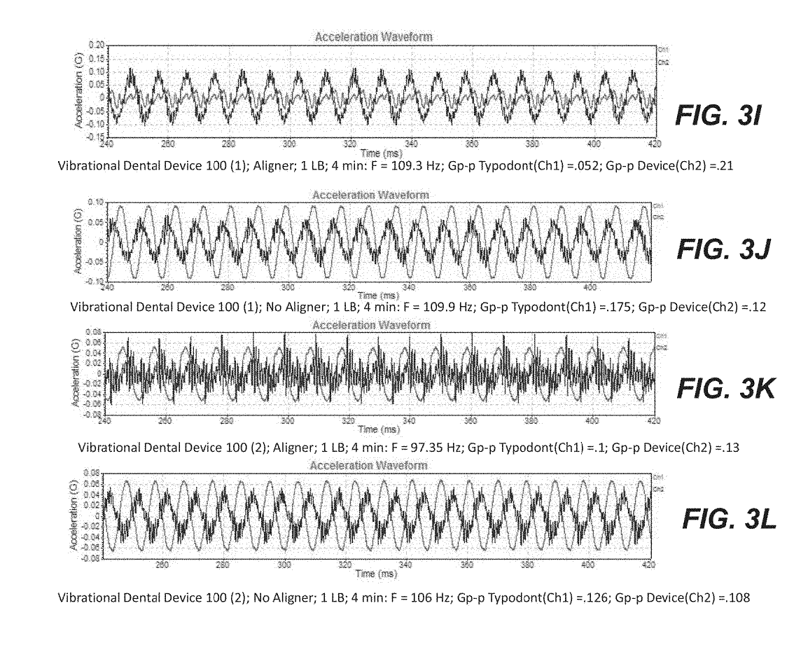

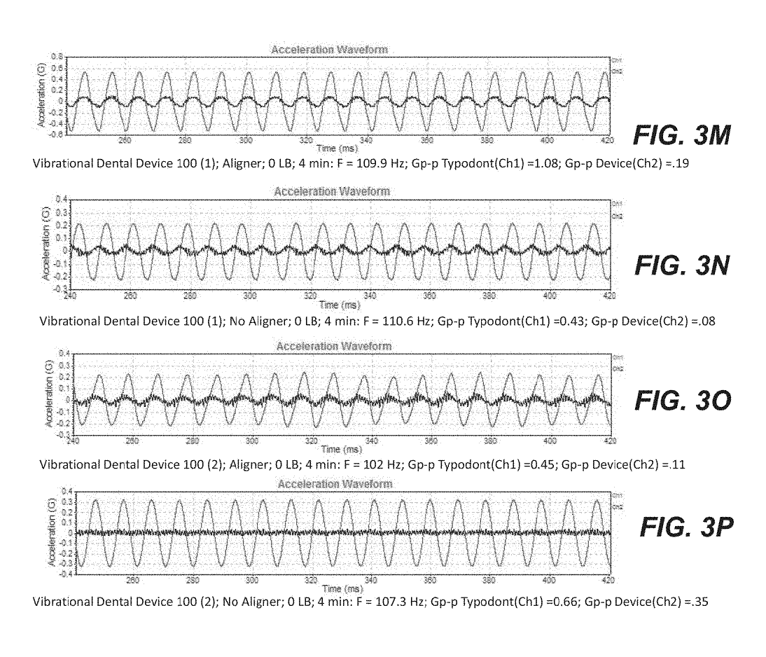

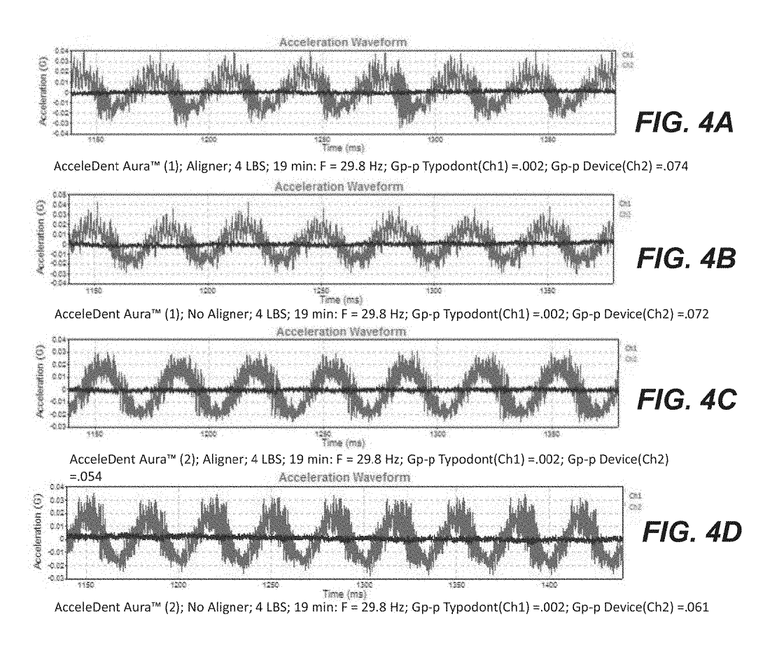

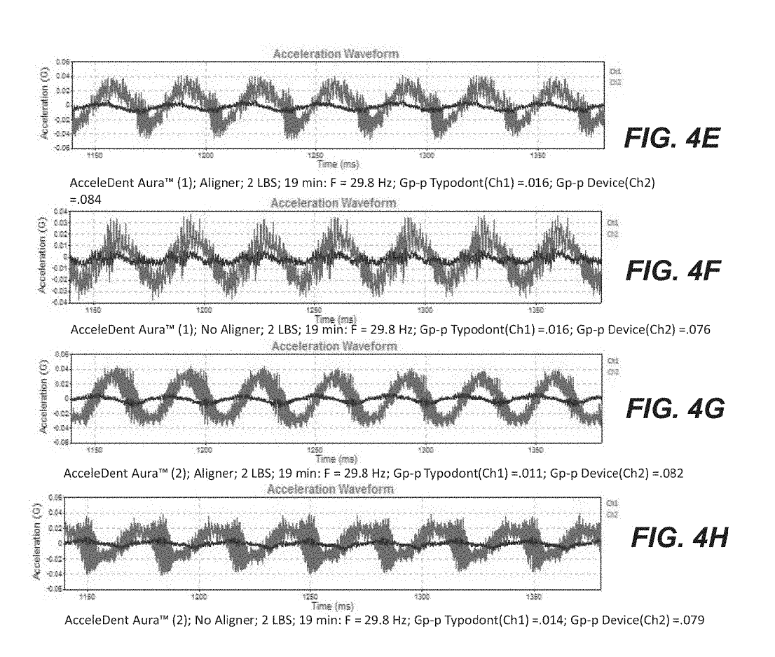

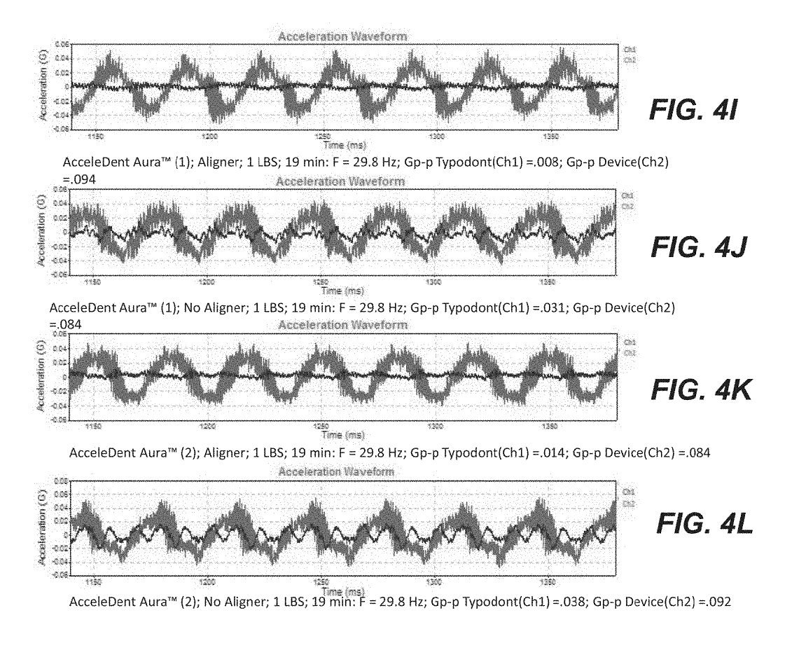

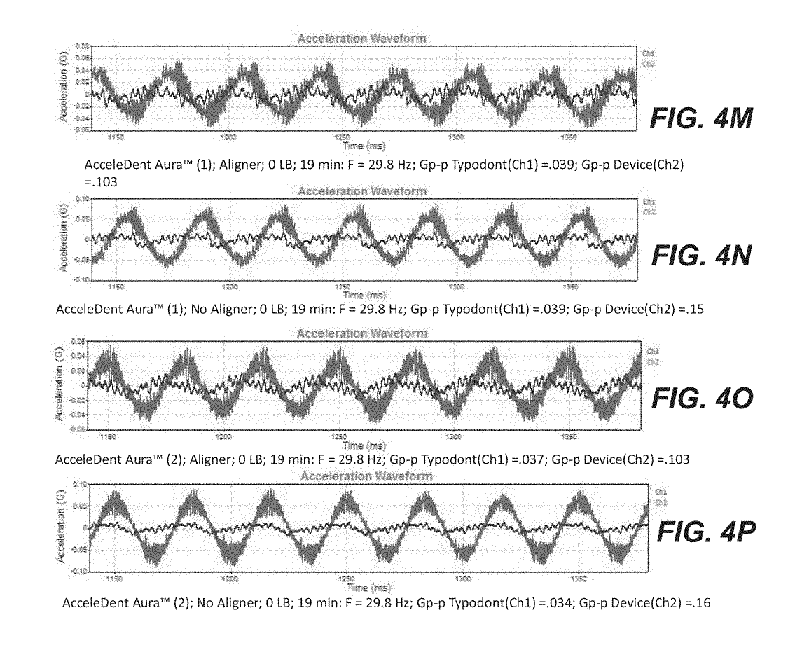

The simulation setup further included electronic instruments, including accelerometers, for measuring vibration characteristics of the typodont. The accelerometers were placed directly on the devices and on the typodont. FIGS. 3A-4P each show the measurement dataset of the accelerometer for two channels, channel 1 (“Ch1”) for detecting the vibration characteristics of the typodont and channel 2 (“Ch2”) for detecting the vibration characteristics of the device. As shown in FIGS. 3A-4P, measurements of the accelerometers over the operation time of each device recorded increasing and decreasing accelerations of the devices and the typodont. The measurement dataset of the accelerometers resembles a sinusoidal curve. The distance from the bottom to the top of the sinusoidal curve is called the peak-to-peak G value or g-force (Gₚ₋ₚ).

In this simulation, the operation time of vibrational dental device 100 was about 5 minutes. The operation time of AcceleDent Aura™ was about 20 minutes. The maximum Gₚ₋ₚ values of the vibration of the typodont actuated by these two devices under different simulated biting forces (different weights) were measured using the accelerometers and other associated electronic instruments about one minute before the end of the operation time. Therefore, measurement of the frequency and g-force for each channel was performed at the time point of about 4 minutes for vibrational dental device 100 and at the time point of about 19 minutes for AcceleDent Aura™.

The simulation was repeated for a second testing device of vibrational dental device 100 and a second testing device of AcceleDent Aura™. Therefore, the first and second vibration dental devices 100 tested are shown as vibrational dental device 100 (1) and vibrational dental device 100 (2) respectively in FIGS. 3A-3P. Also, the first and second AcceleDent Aura™ devices are shown as AcceleDent Aura™ (1) and AcceleDent Aura™ (2) respectively in FIGS. 4A-4P. The simulation was also repeated where the typodont was installed with and without an aligner, as indicated in the captions of FIGS. 3A-4P. All measurement data was summarized in Table 1 as shown below.

TABLE 1
G-force values (Gₚ₋ₚ) measured under different testing conditions.

| | | | | | g-force on | g-force on |
|---|---|---|---|---|---|---|
| | | | | Frequency | typodont | device |
| Device | Weight | Aligner | Time | (Hz) | (G) | (G) |
| AcceleDent Aura ™ (1) | 4 | Yes | End-1 Min | 29.8 | 0.002 | 0.074 |
| AcceleDent Aura ™ (1) | 4 | No | End-1 Min | 30.46 | 0.002 | 0.072 |
| Vibrational dental device 100 (1) | 4 | Yes | End-1 Min | 96.69 | 0.061 | 0.230 |
| Vibrational dental device 100 (1) | 4 | No | End-1 Min | 98.02 | 0.048 | 0.210 |
| AcceleDent Aura ™ (2) | 4 | Yes | End-1 Min | 29.8 | 0.002 | 0.054 |
| AcceleDent Aura ™ (2) | 4 | No | End-1 Min | 29.8 | 0.002 | 0.061 |
| Vibrational dental device 100 (2) | 4 | Yes | End-1 Min | 100 | 0.045 | 0.150 |
| Vibrational dental device 100 (2) | 4 | No | End-1 Min | 98.02 | 0.025 | 0.210 |
| AcceleDent Aura ™ (1) | 2 | Yes | End-1 Min | 29.8 | 0.016 | 0.084 |
| AcceleDent Aura ™ (1) | 2 | No | End-1 Min | 29.8 | 0.016 | 0.076 |
| Vibrational dental device 100 (1) | 2 | Yes | End-1 Min | 104 | 0.045 | 0.150 |
| Vibrational dental device 100 (1) | 2 | No | End-1 Min | 112.6 | 0.081 | 0.150 |
| AcceleDent Aura ™ (2) | 2 | Yes | End-1 Min | 29.8 | 0.011 | 0.082 |
| AcceieDent Aura ™ (2) | 2 | No | End-1 Min | 29.8 | 0.014 | 0.079 |
| Vibrational dental device 100 (2) | 2 | Yes | End-1 Min | 100.7 | 0.033 | 0.125 |
| Vibrational dental device 100 (2) | 2 | No | End-1 Min | 111.3 | 0.046 | 0.118 |
| AcceleDent Aura ™ (1) | 1 | Yes | End-1 Min | 29.8 | 0.008 | 0.094 |
| AcceleDent Aura ™ (1) | 1 | No | End-1 Min | 29.8 | 0.031 | 0.086 |
| Vibrational dental device 100 (1) | 1 | Yes | End-1 Min | 109.3 | 0.052 | 0.210 |
| Vibrational dental device 100 (1) | 1 | No | End-1 Min | 109.9 | 0.175 | 0.120 |
| AcceleDent Aura ™ (2) | 1 | Yes | End-1 Min | 29.8 | 0.014 | 0.084 |
| AcceleDent Aura ™ (2) | 1 | No | End-1 Min | 29.8 | 0.038 | 0.092 |
| Vibrational dental device 100 (2) | 1 | Yes | End-1 Min | 97.35 | 0.100 | 0.130 |
| Vibrational dental device 100 (2) | 1 | No | End-1 Min | 106 | 0.126 | 0.108 |

In exemplary embodiments, the vibrational dental device can be configured to deliver g-forces above those found in prior art devices indicated for use with aligners or without aligners.

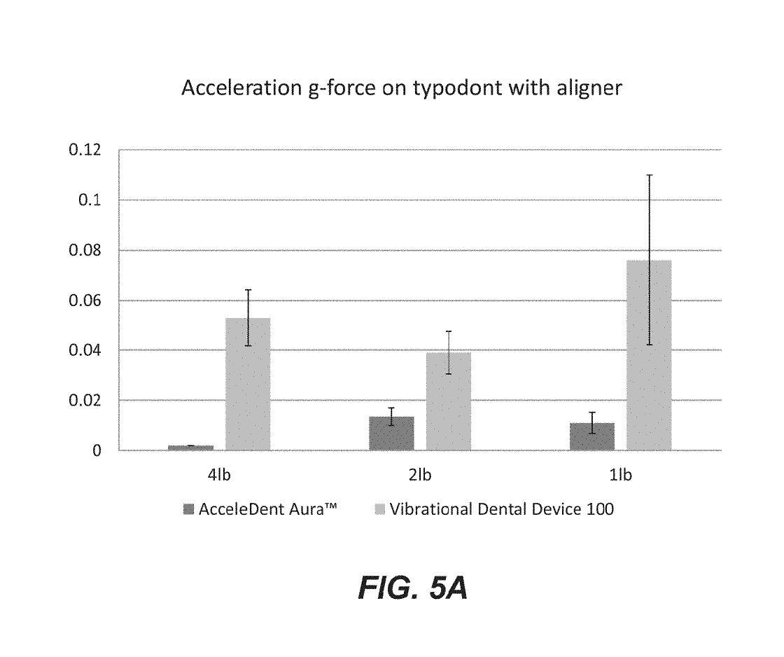

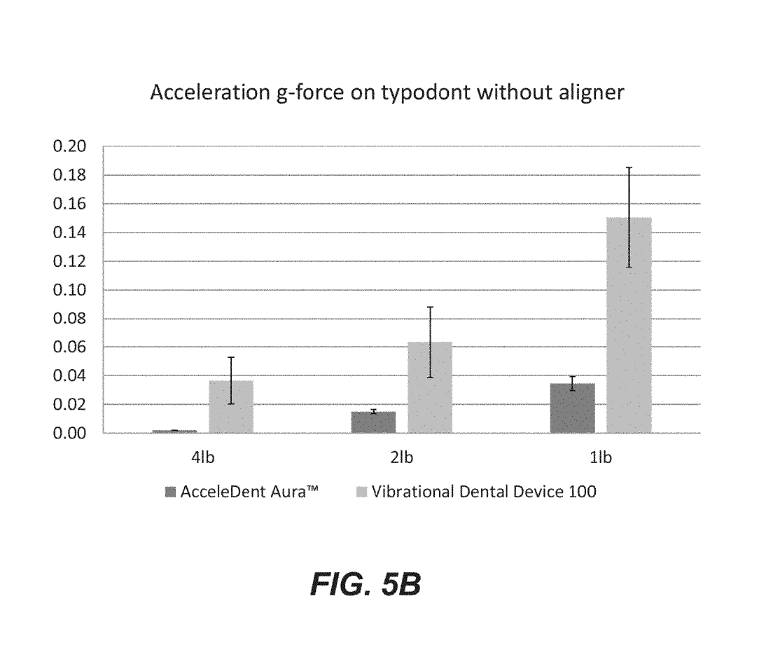

FIG. 5A and Table 2 show the measured g-force values (Gₚ₋ₚ) of the typodont mounted with different weights while subject to vibration by vibrational dental device 100 and by the AcceleDent Aura™ with the aligner. FIG. 5B and Table 3 Shows the measured g-force values (Gₚ₋ₚ) of the typodont mounted with different weights while subject to vibration by vibrational dental device 100 and by the AcceleDent Aura™ without the aligner. As described herein, results shown in FIGS. 5A and 5B and Tables 1 and 2 were average values and standard deviations of the measured g-force values (Gₚ₋ₚ) on the typodont caused by the two testing devices of vibrational dental device 100 and the two testing devices of AcceleDent Aura™.

As shown in FIGS. 5A and 5B, vibrational dental device 100 produced greater acceleration than the AcceleDent Aura™ at various simulated biting forces (under various weights). When the typodont was fitted an aligner (as shown in FIG. 5A and Table 1), depending on the simulated biting force, the AcceleDent Aura™ caused very low acceleration levels of the typodont with g-force values from less than 0.01 G to no greater than 0.02 G. In contrast, vibrational dental device 100 resulted in higher acceleration levels of the typodont with g-force values ranging from about 0.04 G to about 0.076 G. In particular, the two-pound and four-pound weights (or simulated biting force) caused the AcceleDent Aura™'s measured average g-force values to drop to very low levels of 0.0135 G and 0.002 G, respectively. When the typodont was without an aligner (as shown in FIG. 5B and Table 2), depending on the simulated biting force, the AcceleDent Aura™ similarly caused very low acceleration levels with g-force values from less than 0.01 G to no greater than 0.04 G. Again, in contrast, vibrational dental device 100 resulted in multi-fold higher acceleration levels with g-force values ranging from about 0.04 G to about 0.15 G. These results suggest that vibrational dental device 100 can produce greater acceleration magnitude of the typodont under different simulated biting forces than the AcceleDent Aura™ with or without aligners.

TABLE 2
Average g-force values (Gₚ₋ₚ) of the typodont mounted with different
weights while subject to vibration by vibrational dental device
100 and by the AcceleDent Aura ™ with the aligner.

| | 4 lb | | 2 lb | | 1 lb | |
|---|---|---|---|---|---|---|
| Device | Average | SD | Average | SD | Average | SD |
| AcceleDent | 0.002 | 0.0000 | 0.0135 | 0.0035 | 0.011 | 0.0042 |
| Aura ™ | | | | | | |
| Vibrational | 0.053 | 0.0113 | 0.0390 | 0.0085 | 0.076 | 0.0339 |
| dental device | | | | | | |
| 100 | | | | | | |

TABLE 3
Average g-force values (Gₚ₋ₚ) of the typodont mounted with different
weights while subject to vibration by vibrational dental device
100 and by the AcceleDent Aura ™ without the aligner.

| | 4 lb | | 2 lb | | 1 lb | |
|---|---|---|---|---|---|---|
| Device | Average | SD | Average | SD | Average | SD |
| AcceleDent | 0.0020 | 0.0000 | 0.0150 | 0.0014 | 0.0345 | 0.0049 |
| Aura ™ | | | | | | |
| Vibrational | 0.0365 | 0.0163 | 0.0635 | 0.0247 | 0.1505 | 0.0346 |
| dental device | | | | | | |
| 100 | | | | | | |

### Example 2

Vibrational dental device 100 and AcceleDent Aura™ also produce distinct vibration frequencies and apply different treatment duration, but they produce about the same number of oscillations per treatment. In this example, cell proliferation of cultured cell samples was quantitatively evaluated and used as a marker for cells' responsivity to the mechanical vibration generated by these two devices. The effects of mechanical vibration on cell proliferation caused by these two devices were then compared.

Commercially available human osteoblasts and human periodontal ligament fibroblasts (Lonza, Inc., Walkersville, Md.) were cultured according to the manufacturer's instructions. For all experiments, the cells were plated in multi-well tissue culture plates at a cell density of 7,500 cells/cm² and placed in an incubator prior to vibration treatment. The cells were taken out from the incubator immediately prior to vibration treatment. Osteoblast and fibroblast cell samples were respectively separated into three groups, a first group subject to vibration treatment by AcceleDent Aura™, a second group subject to vibration treatment by an exemplary embodiment of vibrational dental device 100, and a third control group handled identically to the other two groups but not subject to vibration treatment. Vibration treatment was applied by placing the device between the cell culture plates and a plastic box that isolated the vibrations generated by the device from being transmitted to the experimental countertop. The cell culture plates, the device, and the plastic box were securely fastened together.

Vibration treatment of the cells was applied daily at room temperature for a period of three days. Each day, the first groups of osteoblasts and fibroblasts were subject to vibration treatment by AcceleDent Aura™ at a frequency of 30 Hz for 20 minutes. The second groups of osteoblasts and fibroblasts were subject to vibration treatment by vibrational dental device 100 at a frequency from about 100 Hz to about 120 Hz for 5 minutes and subsequently left at room temperature for 15 minutes to match the 20 minutes room temperature exposure of the first groups. The control groups were left at room temperate for 20 minutes without any vibration treatment.

Cell density (cells/cm²) of the three groups of osteoblasts and fibroblasts was determined over three days. A standard spectrophotometric MTS assay was used for determining cell density according to the manufacturer's instructions (XTT Assay, ATCC™). The cell sample size for each group was 5. Mean cell numbers of each group were calculated based on the measured cell densities of the cell samples of each group. The mean cell numbers of the three groups of osteoblasts and fibroblasts were statistically compared to each other via Fisher tests. A significance value of 0.05 was used for all datasets.

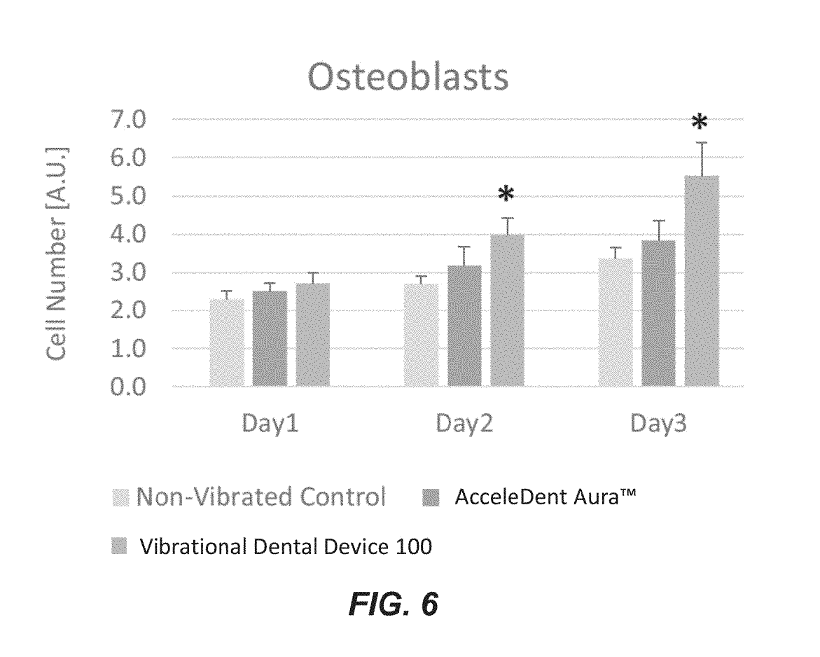

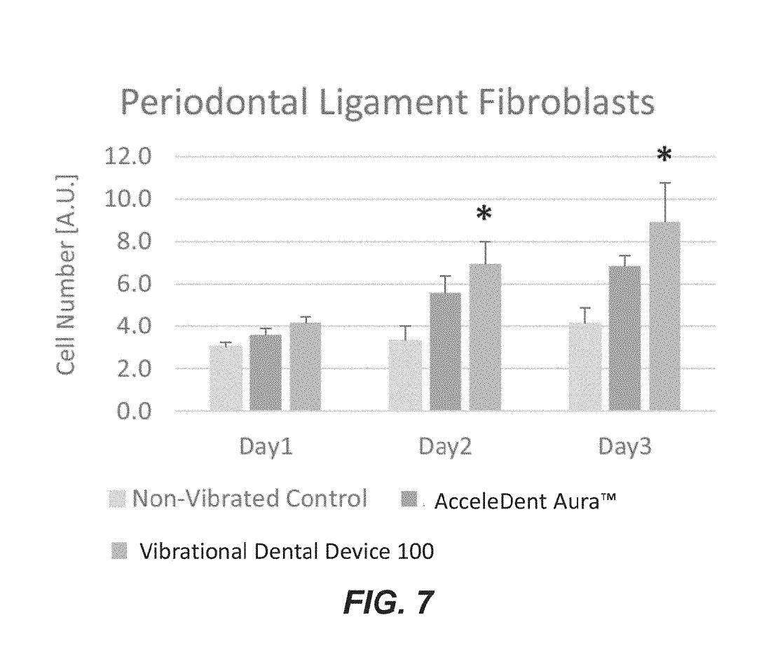

FIG. 6 shows mean cell numbers of three groups of osteoblasts. FIG. 7 shows mean cell numbers of three groups of fibroblasts. The cell numbers are presented in an arbitrary unit for the purpose of comparison. Surprisingly, as shown in FIG. 6, the group of osteoblasts subject to vibration treatment by vibrational dental device 100 had significantly greater (p<0.05) cell proliferation than the group of osteoblasts subject to vibration treatment by AcceleDent Aura™ on day 2 and day 3. Similarly, as shown in FIG. 7, the group of fibroblasts subject to vibration treatment by vibrational dental device 100 had significantly greater (p<0.05) cell proliferation than the group of fibroblasts subject to vibration treatment by AcceleDent Aura™ on day 2 and day 3.

This example demonstrates the surprising results that vibration treatment by vibrational dental device 100 enhanced proliferation of human osteoblasts and periodontal ligament fibroblasts to a significantly greater level than AcceleDent Aura™. The in vitro experimental results presented in this example suggest that different cell types and tissues, such as the human osteoblasts and human periodontal ligament fibroblasts, can be more responsive to the vibration variables used by vibrational dental device 100, e.g., at a frequency from about 100 Hz to about 120 Hz and a duration of only about 5 minutes, than the vibration variables used by AcceleDent Aura™, e.g., at a frequency of 30 Hz and a duration of 20 minutes. That superior results could be obtained in one-fourth the time required by the AcceleDent Aura™ could result in superior clinical results in orthodontics, as shorter treatment periods generally correlate to better user compliance and adherence.

### Example 3

An exemplary embodiment of vibrational dental device 100 is used to subject periodontal cells for a period of vibrational treatment. The periodontal cells include human osteoblasts in alveolar bone and periodontal ligament fibroblasts. The periodontal cells are treated for less than about 20 minutes, for example for about 5 minutes, at a frequency higher than about 80 Hz, for example from about 100 Hz to about 120 Hz, daily over a period of time, which lasts for about a few days to a couple of weeks. At the end of the period of time, the number of periodontal cells, including osteoblasts in alveolar bone and periodontal ligament fibroblasts, is increased.

### Example 4

An exemplary embodiment of vibrational dental device 100 is used to subject periodontal cells for a period of vibrational treatment. The periodontal cells include human osteoblasts in alveolar bone and periodontal ligament fibroblasts. The periodontal cells are treated for less than about 20 minutes, for example for about 5 minutes, at a frequency higher than about 80 Hz, for example from about 100 Hz to about 120 Hz, daily over a period of time, which lasts for about a couple of weeks to about a month. At the end of the period of time, the number of periodontal cells, including osteoblasts in alveolar bone and periodontal ligament fibroblasts, is increased.

### Example 5

An exemplary embodiment of vibrational dental device 100 is used to subject periodontal cells for a period of vibrational treatment. The periodontal cells include human osteoblasts in alveolar bone and periodontal ligament fibroblasts. The periodontal cells are treated for less than about 20 minutes, for example for about 5 minutes, at a frequency higher than about 80 Hz, for example from about 100 Hz to about 120 Hz, daily over a period of time, which lasts for about a month to a couple of months. At the end of the period of time, the number of periodontal cells, including osteoblasts in alveolar bone and periodontal ligament fibroblasts, is increased.

### Example 6

An exemplary embodiment of vibrational dental device 100 is used to subject periodontal cells for a period of vibrational treatment. The periodontal cells include human osteoblasts in alveolar bone and periodontal ligament fibroblasts. The periodontal cells are treated for less than about 20 minutes, for example for about 5 minutes, at a frequency higher than about 80 Hz, for example from about 100 Hz to about 120 Hz, daily over a period of time, which lasts for about a couple of months to about a few months. At the end of the period of time, the number of periodontal cells, including osteoblasts in alveolar bone and periodontal ligament fibroblasts, is increased.

### Example 7

An exemplary embodiment of vibrational dental device 100 is provided to a user for a period of vibrational treatment. While wearing an orthodontic aligner, the user uses vibrational dental device 100 by clamping the mouthpiece 102 between his or her teeth for less than about, for example about 5 minutes at a frequency higher than about 80 Hz, for example from about 100 Hz to about 120 Hz daily, during the period of vibrational treatment. The period of vibrational treatment lasts for about 2 days to about a few days. At the end of the period of vibrational treatment, the number of periodontal cells, including osteoblasts in alveolar bone and periodontal ligament fibroblasts, of the teeth that are subject to the vibrational treatment by vibrational dental device 100 is increased. Additionally, a desired movement of all or some of the teeth subject to vibrational treatment by vibrational dental device 100 is achieved at the end of the period of vibrational treatment.

### Example 8

An exemplary embodiment of vibrational dental device 100 is provided to a user for a period of vibrational treatment. While wearing an orthodontic aligner, the user uses vibrational dental device 100 by clamping the mouthpiece 102 between his or her teeth for less than about 20 minutes, for example for about 5 minutes, at a frequency from higher than about 80 Hz, for example about 100 Hz to about 120 Hz, daily during the period of vibrational treatment. The period of vibrational treatment lasts for about a few days to a couple of weeks. At the end of the period of vibrational treatment, the number of periodontal cells, including osteoblasts in alveolar bone and periodontal ligament fibroblasts, of the teeth that are subject to the vibrational treatment by vibrational dental device 100 is increased. Additionally, a desired movement of all or some of the teeth subject to vibrational treatment by vibrational dental device 100 is achieved at the end of the period of vibrational treatment.

### Example 9

An exemplary embodiment of vibrational dental device 100 is provided to a user for a period of vibrational treatment. While wearing an orthodontic aligner, the user uses vibrational dental device 100 by clamping the mouthpiece 102 between his or her teeth for less than about 80 Hz, for example for about 5 minutes, at a frequency higher than about 80 Hz, for example from about 100 Hz to about 120 Hz, daily during the period of vibrational treatment. The period of vibrational treatment lasts for about a couple of weeks to about a month. At the end of the period of vibrational treatment, the number of periodontal cells, including osteoblasts in alveolar bone and periodontal ligament fibroblasts, of the teeth that are subject to the vibrational treatment by vibrational dental device 100 is increased. Additionally, a desired movement of all or some of the teeth subject to vibrational treatment by vibrational dental device 100 is achieved at the end of the period of vibrational treatment.

An exemplary embodiment of vibrational dental device 100 is provided to a user for a period of vibrational treatment. While wearing an orthodontic aligner, the user uses vibrational dental device 100 by clamping the mouthpiece 102 between his or her teeth for less than about 20 minutes, for example for about 5 minutes, at a frequency higher than about 80 Hz, for example from about 100 Hz daily to about 120 Hz during the period of vibrational treatment. The period of vibrational treatment lasts for about a month to a couple of months. At the end of the period of vibrational treatment, the number of periodontal cells, including osteoblasts in alveolar bone and periodontal ligament fibroblasts, of the teeth that are subject to the vibrational treatment by vibrational dental device 100 is increased. Additionally, a desired movement of all or some of the teeth subject to vibrational treatment by vibrational dental device 100 is achieved at the end of the period of vibrational treatment.

### Example 11

An exemplary embodiment of vibrational dental device 100 is provided to a user for a period of vibrational treatment. While wearing an orthodontic aligner, the user uses vibrational dental device 100 by clamping the mouthpiece 102 between his or her teeth for less than about 20 minutes, for example for about 5 minutes, at a frequency higher than about 80 Hz, for example from about 100 Hz to about 120 Hz, daily during the period of vibrational treatment. The period of vibrational treatment lasts for about a couple of months to a few months. At the end of the period of vibrational treatment, the number of periodontal cells, including osteoblasts in alveolar bone and periodontal ligament fibroblasts, of the teeth that are subject to the vibrational treatment by vibrational dental device 100 is increased. Additionally, a desired movement of all or some of the teeth subject to vibrational treatment by vibrational dental device 100 is achieved at the end of the period of vibrational treatment.

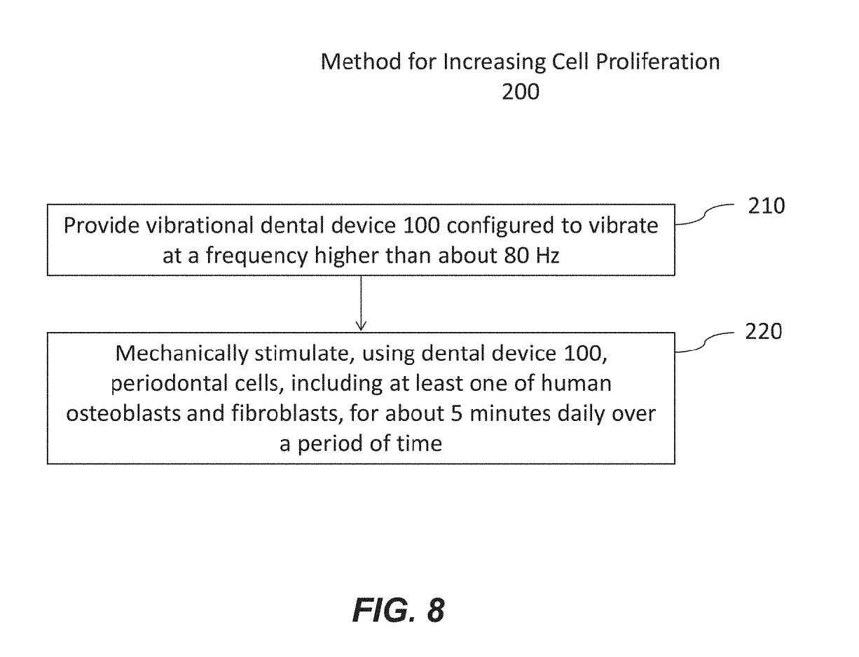

The above-described examples suggest that vibrational dental device 100 described herein may be utilized in a variety of procedures and methods for increasing cell proliferation. An exemplary method 200 for increasing cell proliferation may use one or more features of the embodiments of vibrational dental device 100, described above in reference to FIGS. 1-5. Exemplary embodiments of method 200 are described below with reference to FIG. 8.

As shown in FIG. 8, method 200 may include steps 210-230. Step 210 may include providing vibrational dental device 100. As described above, while in operation, vibrational dental device 100 can be configured to vibrate at a frequency higher than about 80 Hz and an acceleration magnitude ranging between about 0.03 G and about 0.2 G. For example, vibrational dental device 100 may vibrate at a frequency between about 100 Hz and about 120 Hz.

Step 220 may include mechanically stimulating, using vibrational dental device 100, cells for about less than 20 minutes, for example for about 5 minutes, daily over a period of time. The cells may include one of human osteoblasts and fibroblasts. As described herein, the period of time may last until a desirable result has achieved. In some embodiments, the period of time may last for a couple of days up to a few months.

Step 230 may include increasing the number of the cells at the end of the period of time. As described herein, an increase of the number of cells may be represented by an increase of the cell density, which indicates cell proliferation. Step 230 may further include quantitatively evaluating the cell number and/or cell density daily.

As described herein, additional steps may be added to method 200. For example, method 200 may include increasing or decreasing the frequency and/or acceleration magnitude of vibrational dental device 100 based on the response of the cells to vibration treatment. Method 200 may also include detecting characteristics of the vibration produced by vibrational dental device 100 and/or characteristics of the vibration applied to the cells by vibrational dental device 100. Also, some steps may be omitted or repeated, and/or may be performed simultaneously.

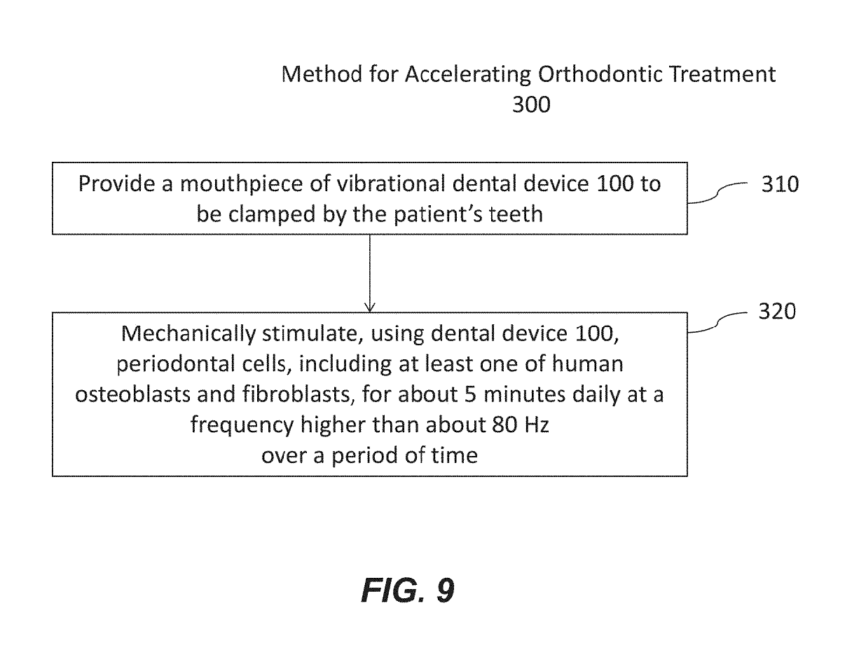

The above-described examples may further suggest that vibrational dental device 100 may stimulate more response of human osteoblasts and periodontal ligament fibroblasts during orthodontic treatment than AcceleDent Aura™. Therefore, vibrational dental device 100 described herein may be utilized in a variety of procedures and methods for accelerating orthodontic tooth movement in orthodontic treatments. An exemplary method 300 for accelerating orthodontic tooth movement may use one or more features of the embodiments of vibrational dental device 100, described above in reference to FIGS. 1-5. Exemplary embodiments of method 300 are described below with reference to FIG. 9.

As shown in FIG. 9, method 300 may include steps 310 and 320. Step 310 may include providing a mouthpiece of vibrational dental device 100 between the occlusal surfaces of a user's teeth to be clamped by the user's teeth. As described above, while in operation, vibrational dental device 100 is capable of vibrating at a frequency higher than about 80 Hz and an acceleration magnitude ranging between about 0.03 G and about 0.2 G. For example, vibrational dental device 100 may vibrate at a frequency between about 100 Hz and 120 Hz.

Step 320 may include mechanically stimulating, using vibrational dental device 100, cells of the user for less than about 20 minutes, for example for about 5 minutes, daily at a frequency higher than 80 Hz over a period of time. The cells may include one of human osteoblasts and periodontal ligament fibroblasts. As described herein, the period of time may last until a desirable result has been achieved, such as a desired amount of tooth movement. In some embodiments, the period of time may last for a few of days up to a few months.

As described herein, additional steps may be added to method 300. For example, method 300 may include increasing or decreasing the frequency and/or acceleration magnitude of vibrational dental device 100 based on the response of vibration treatment, such as the speed or amount of tooth movement. Method 300 may also include detecting characteristics of the vibration applied proximate to the occlusal surfaces, such as frequency and acceleration magnitude, by sensors of vibrational dental device 100. Also, some steps may be omitted or repeated, and/or may be performed simultaneously.

The foregoing description has been presented for purposes of illustration. It is not exhaustive and is not limited to precise forms or embodiments disclosed. Modifications and adaptations of the embodiments will be apparent from consideration of the specification and practice of the disclosed embodiments. For example, the described implementations include hardware and software, but systems and methods consistent with the present disclosure can be implemented as hardware alone. In addition, while certain components have been described as being coupled to one another, such components may be integrated with one another or distributed in any suitable fashion.

Moreover, while illustrative embodiments have been described herein, the scope includes any and all embodiments having equivalent elements, modifications, omissions, combinations (e.g., of aspects across various embodiments), adaptations and/or alterations based on the present disclosure. The elements in the claims are to be interpreted broadly based on the language employed in the claims and not limited to examples described in the present specification or during the prosecution of the application, which examples are to be construed as nonexclusive. Further, the steps of the disclosed methods can be modified in any manner, including reordering steps and/or inserting or deleting steps.

The features and advantages of the disclosure are apparent from the detailed specification, and thus, it is intended that the appended claims cover all systems and methods falling within the true spirit and scope of the disclosure. As used herein, the indefinite articles “a” and “an” mean “one or more.” Similarly, the use of a plural term does not necessarily denote a plurality unless it is unambiguous in the given context. Words such as “and” or “or” mean “and/or” unless specifically directed otherwise. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

Other embodiments will be apparent from consideration of the specification and practice of the embodiments disclosed herein. It is intended that the specification and examples be considered as example only, with a true scope and spirit of the disclosed embodiments being indicated by the following claims.

What is claimed is:

1. A method for increasing periodontal cell proliferation, the method comprising:
providing a vibrational dental device configured to vibrate at a frequency higher than about 80 Hz;
mechanically stimulating, using the vibrational dental device, periodontal cells, including at least one of human osteoblasts and fibroblasts, for about 5 minutes daily over a period of time; and
wherein the number of the periodontal cells at the end of the period of time is increased.

2. The method of claim 1, wherein the vibrational dental device is configured to vibrate at an acceleration magnitude ranging between about 0.03 G and about 0.2 G.

3. The method of claim 1, further comprising mechanically stimulating the cells at a frequency between about 100 Hz and about 120 Hz.

4. The method of claim 1, further comprising mechanically stimulating the cells at a g-force between about 0.03 G and about 0.2 G.

5. The method of claim 1, wherein the vibrational dental device comprises a mouthpiece and a motor connected to and configured to vibrate the mouthpiece.

6. The method of claim 5, further comprising providing the mouthpiece between the occlusal surfaces of a user's teeth to be clamped by the user's teeth.

7. The method of claim 6, further comprising applying an axial vibratory force on the occlusal surfaces by the vibrational dental device.

8. The method of claim 1, further comprising detecting vibration characteristics of the mouthpiece.

9. The method of claim 1, further comprising adjusting the frequency and/or g-force of the vibration of the vibrational dental device.

10. The method of claim 1, wherein the period of time ranges from a couple of days to a few months.

11. The method of claim 10, wherein the period of time ranges from a couple of days to a few days.

12. The method of claim 10, wherein the period of time ranges from a few days to couple of weeks.

13. The method of claim 10, wherein the period of time ranges from a month to a couple of months.

14. The method of claim 10, wherein the period of time ranges from a couple of months to a few months.

15. The method of claim 1, wherein the human osteoblasts comprise human osteoblasts in alveolar bone.

16. The method of claim 1, wherein the fibroblasts comprise periodontal ligament fibroblasts.

17. A method for accelerating orthodontic tooth movement, the method comprising:
providing a mouthpiece of a vibrational dental device to be clamped by the user's teeth; and
mechanically stimulating, using the vibrational dental device, cells of the user, including at least one of osteoblasts in alveolar bone and periodontal ligament fibroblasts, for about 5 minutes daily at a frequency higher than about 80 Hz over a period of time.

18. The method of claim 17, further comprising providing the mouthpiece between the occlusal surfaces of a user's teeth.

19. The method of claim 17, further comprising mechanically stimulating the cells of the user at a g-force between about 0.03 G and about 0.2 G.

20. The method of claim 17, further comprising mechanically stimulating the cells of the user at a frequency between about 100 Hz and about 120 Hz.

21. A dental device for increasing cell proliferation, the dental device comprising means for
mechanically stimulating cells, including at least one of human osteoblasts and periodontal ligament fibroblasts, at a frequency higher than about 80 Hz for about 5 minutes daily over a period of time; and
increasing the number of the cells at the end of the period of time.
